(19) United States
(12) Patent Application Publication
(10) Pub. No.: US 2023/0256043 A1
(43) Pub. Date: Aug. 17, 2023

(54) FORMULATION PRODUCT

(71) Applicants: The University Court of the University of Aberdeen, Aberdeenshire (GB); Nutriigen Ltd., Glasgow (GB)

(72) Inventors: Madalina NEACSU, Aberdeen (GB); Wendy Roslyn RUSSELL, Aberdeen (GB); Nigel Edmond YOUNG, Glasgow (GB); Alan Michael ROWE, Glasgow (GB); Victor THOMSON, Glasgow (GB)

(73) Assignees: The University Court of the University of Aberdeen, Aberdeenshire (GB); Nutriigen Ltd., Glasgow (GB)

(21) Appl. No.: 18/015,952

(22) PCT Filed: Jul. 13, 2021
(86) PCT No.: PCT/GB2021/051799

§ 371 Date: Jan. 12, 2023

(30) Foreign Application Priority Data
Jul. 13, 2020 (GB) 2010755.3
Mar. 15, 2021 (GB) 2103580.3

(51) Int. Cl. A61K 36/185; A61K 9/50; A61K 9/48; A23P 10/30; A23P 10/40; A23L 33/105; A23L 29/256; A23L 29/25; A23L 29/30; A23L 29/262

(52) U.S. Cl. CPC A61K 36/185; A61K 9/5036; A61K 9/5089; A61K 9/4866; A61K 9/4833; A23P 10/30; A23P 10/40; A23L 33/105; A23L 29/256; A23L 29/25; A23L 29/35; A23L 29/262; A61K 2236/17

(57) ABSTRACT

The present invention relates to the field of food technology and in particular, nutrient and bioactive rich plant formulations. Formulated products including nutrients and bioactives such as those provided by the Moringa plant are provided. In particular, coated or encapsulated food products having a spec release behaviour and a process for their production is described. The processes described enable food material, for example Moringa, to be used to provide nutrient dense foods that are more accessible and protected against loss of nutritional value. The resulting food products therefore allow better delivery of nutritional benefits to users.

### FIELD OF THE INVENTION

The present invention relates to the field of food technology and in particular, nutrient and bioactive rich plant formulations, suitably to provide formulated products including nutrients and bioactives such as those provided by the Moringa plant.

### BACKGROUND

Micronutrients are vital to development, disease prevention and well-being. In animals, for example humans, key micronutrients required for health cannot be produced in the body and must be derived from the diet. At least half of all children worldwide aged 6 months to five years suffer from one or more micronutrient deficiencies and globally more than two million are affected from micronutrient deficiencies.

Moringa is a plant native to parts of Africa and Asia. It is the sole genus of the flowering plant family Moringacea. Moringa oleifera is the most widely cultivated species of the genus Moringa. The Moringa tree, also known as the drumstick tree, horseradish tree, benoil tree or benzoil tree is known to have edible parts, in particular the pods, leaves, seeds and roots. In particular, Moringa leaves are known to be rich in protein (around 27% per weight) and these leaves can form an integral part of the diet in some parts of the world. Moringa leaves are considered to provide a range of vitamins and minerals. Moringa leave are also considered to contain a good balance of amino acids. Typically, in use, prior to being eaten the leaves are boiled. Moringa leaf powder is also known for use in sauces or as a general food additive.

It would be beneficial if the nutritional benefits of Moringa based food products could be made accessible by designing formulated products with improved palatability.

### SUMMARY OF INVENTION

Whilst plant formulations such as the Moringa plant can provide nutritional benefits, it is considered that these benefits could be improved. For example, the nutritional benefits could be provided as part of specialised formulated products. These products may contain whole plant or whole parts or extract thereof (i.e. whole leaf, seed, pod, root, stalk,etc) and provide for example for an enhanced taste, or the ability for nutrients to be released to certain sites and/or with an increased shelf life.

Moringa is understood to have a distinctive taste and for some uses it may be advantageous to provide Moringa or a part thereof such that the taste of Moringa is masked. For example, it is understood that the isothiocyanates present in Moringa can provide a pungent taste and it may be appropriate to mask the taste provided by these isothiocyanates. The taste from isothiocyanates may be masked, for example, by minimising the presence of isothiocyanate or by masking the isothiocyanate from the taste buds or reducing the release of isothiocyanates in the mouth when Moringa is consumed.

Accordingly, there is provided a process to encapsulate or microencapsulate plant material the process comprising the steps of:
mixing milled plant material with an encapsulation agent selected from:
a) a plant material: encapsulation agent at a ratio of 1:1 w/w to encapsulate the plant material; or
b) a plant material: coating for microencapsulation at a ratio of 1:2 to 1:4 w/w;
to provide a homogenized mixture wherein the plant material is encapsulated by the encapsulating agent or coated with the microencapsulation agent, and optionally further processing the homogenized mixture.

Suitably material which may be used as an encapsulation agent may be selected from: alginate, carrageen, cellulose sulphate, chitosan, gelatine and pectin, as well as waxes.

Suitably, coating for microencapsulation may be water soluble polysaccharides (cellulosic and non cellulosic) and non polysaccharides. Suitably examples comprise: Guar gum, Carrageenan, Sodium alginate, Chitosan, Gum Arabic, Karaya gum, methylcellulose, sodium carboxylmethyl cellulose, hydroxypropyl methylcellose, Gelatin, polyvinyl alcohol, polyethylene glycol. Another category are non-water soluble coating materials: organic soluble polymers, latex, dispersions, pseudo latex, and non-polymers.

Suitably, a plant material: coating for microencapsulation is provided at a ratio of 1:2 to 1:4 w/w;

to provide a homogenized mixture wherein the plant material is encapsulated by the microencapsulation agent may be further processed by spray drying.

Suitably, the plant material may be Moringa plant material and the Moringa plant material may be milled Moringa plant material with an encapsulation agent selected from:
Alginate—at a Moringa: Alginate ratio of 1:1 w/w.

Alternatively, the plant material may be Moringa plant material and the Moringa plant material may be milled Moringa plant material with a coating for microencapsulation
Arabic gum—at a Moringa: Arabic gum ratio of 1:2 to 1:4 w/w; or
Arabic gum maltodextrin—at a Moringa:Arabic gum: maltodextrin ratio of 1:2:1
to provide a homogenized mixture wherein the Moringa is encapsulated by the encapsulating agent.

Suitably Moringa may be milled and encapsulation may be achieved using Biotech encapsulator: frequency 1000 Hz, electrode 2210 air control 700 ml/h. Suitably bead formation may be in the range of 0.15 mm to 2.0 mm.

Suitably milling of plant material may be undertaken to provide milled plant material, suitably Moringa plant material, in the range of 2 micrometers to 1000 micrometers, suitably 2 to 500 micrometers, suitably 2 to 300 micrometers, suitably at least 50 micrometers, suitably at least 100 micrometers. The size of the milled plant material, suitably Moringa, may be dictated by the nozzle of a spray drying microencapsulation system wherein the milled material should be sized below the nozzle diameter.

It is considered a homogenized mixture is a mixture that is stable for the period during which the mixture is further processed, for example when the mixture is spray dried. Suitably the mixture may be stable for a couple of hours until overnight (for example 2 hours to at least 16-18 hour), or more if the consistency of material is more viscous.

The homogenised Moringa may be further processed, for example to provide the mixture in a different form, for example a powder form wherein:

a) Moringa: Encapsulation agent (for example Alginate) 1:1 mixture is provided in a CaCl₂) hardening bath;

b) Moringa: coating for microencapsulation (for example Arabic gum mixture, or

Moringa: Arabic gum: Maltodextrin) mixture is spray dried or the like (atomization at room temperature).

Suitably the hardening bath may be a CaCl₂) (6%) hardening bath.

Suitably further processing of the homogenized mixture may be by microencapsulation techniques as: Spray drying, Fluidized bed dryer (air suspension coating), Coacervation, Pan coating, Spray Congealing, Solvent evaporation, Polymerisation, or Centrifugal process. However, the technological process needs to be adapted accordingly for each individual technique.

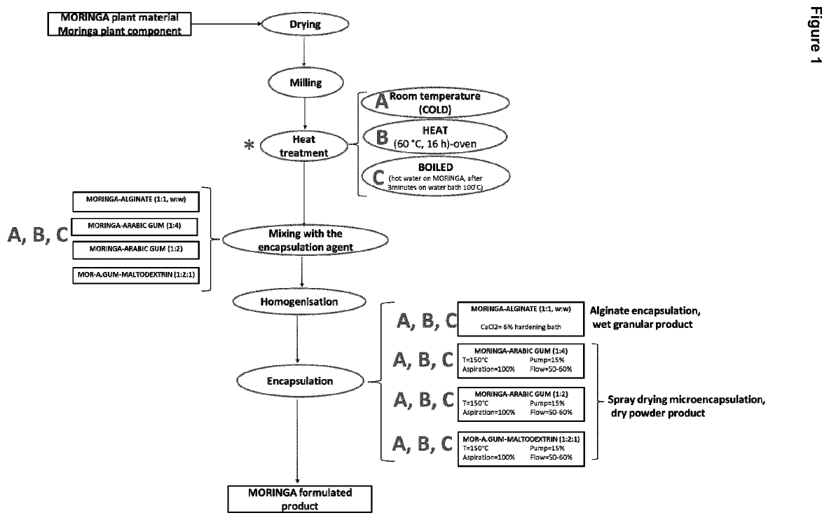

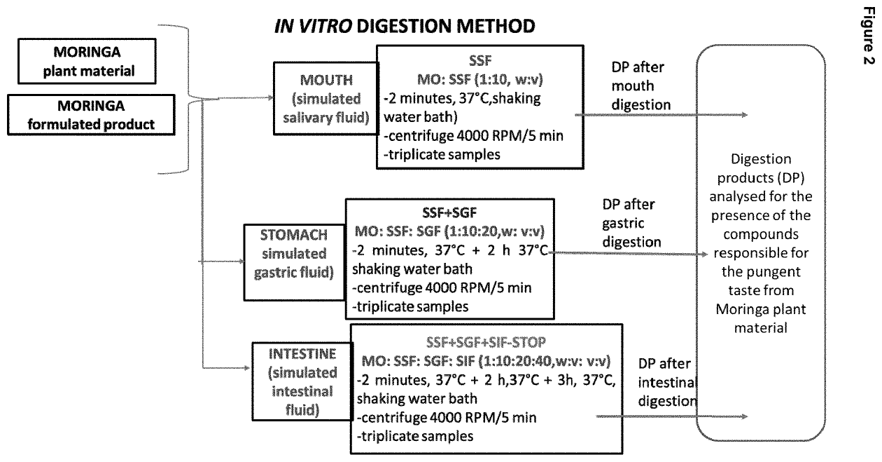

An example of the process is provided by FIG. 1.

Suitably, spray drying using plant material:microencapsulating agent (for example Moringa: microencapsulating agent) described uses a temperature of 150° C., flow at 50-60%, Aspiration of 100%.

Suitably, the plant material used the process, for example Moringa used in the process may be whole plant or a component (i.e. protein fraction, fiber enriched fraction or bioactive phytochemicals rich fraction). Suitably, the process may include a pre-treatment step, after milling and before mixing.

Suitably, the pre-treatment may comprise storage of the milled material at room temperature.

Suitably, heat treatment may be by heating the plant material, for example Moringa material to at least 60 degrees Celsius for at least 10 hours, at least 15 hours at least hours, suitably 16 hours.

Suitably, the pre-treatment step may comprise heating of the milled material to 60° C. for 16 hours in an oven.

Alternatively, heat treatment prior to the step of homogenizing may be boiling of the Moringa in a water bath. Suitably the water bath may be 100 degrees Celsius and boiling will be for at least two, at least three, at least five minutes. Suitably, the pre-treatment may be providing hot water to the Moringa, for example 3 minutes in a water bath at 100° C.

Examples of, microencapsulation using a pre-treatment step as indicated and encapsulating agent is illustrated in FIG. 1.

Advantageously, the inventors have determined that the process of the present invention may be undertaken and a yield of microencapsulated Moringa can be achieved of at least 60%, at least 70%. Suitably a yield about 74% may be achieved as set out in Table 1.

TABLE 1

| Process including pretreatment | Encapsulating agent used and yield |
|---|---|
| step | obtained |
| COLD (room | MO:AG (1:4) => η = 74% |
| temperature) | MO:AG:MT (1:2:1) => η = 72.5% |
| HEAT TREATMENT | MO:AG (1:4) => η = 70% |
| (60° C., 16 h)- oven | MO:AG:MT (1:2:1) => η = 67.5% |
| BOILED | MO:AG (1:4) => η = 74% |
| | MO:AG:MT (1:2:1) => η = 72.5% |

MO: Moringa plant material;
AG: Arabic gum;
MT: maltodextrin

Without wishing to be bound by theory, the inventors consider the microencapsulation process impedes the release in the mouth of potential molecules responsible for the pungent taste of Moringa. This (the microencapsulation efficiency for masking the Moringa plant material taste) has been tested by simulation the formulated product digestion in laboratory.

Moreover, the encapsulation process is also considered to provide an environment for the Moringa to maximise the release of the formulated product components in the stomach and small intestine.

According to a second aspect of the present invention there is provided a nutritional composition comprising encapsulated plant material, for example Moringa, as provided by the process of the first aspect of the invention.

Suitably the encapsulated plant material (for example Moringa) may provide substantially spherical product of size in the range 200 micrometers to about 1 cm in diameter, suitably at least 300, suitably at least 400, suitably at least 500, suitably at least 600 micrometers.

Suitably plant pigments, aromas, spices etc may be provided with the encapsulated or microencapsulated material.

Preferred features and embodiments of each aspect of the invention are as for each of the other aspects mutatis mutandis unless context demands otherwise.

Each document, reference, patent application or patent cited in this text is expressly incorporated herein in their entirety by reference, which means it should be read and considered by the reader as part of this text. That the document, reference, patent application or patent cited in the text is not repeated in this text is merely for reasons of conciseness.

Reference to cited material or information contained in the text should not be understood as a concession that the material or information was part of the common general knowledge or was known in any country.

As used herein, the articles “a” and “an” refer to one or to more than one (for example to at least one) of the grammatical object of the article.

“About” shall generally mean an acceptable degree of error for the quantity measured given the nature or precision of the measurements.

Throughout the specification, unless the context demands otherwise, the terms ‘comprise’ or ‘include’, or variations such as ‘comprises’ or ‘comprising’, ‘includes’ or ‘including’ will be understood to imply the includes of a stated integer or group of integers, but not the exclusion of any other integer or group of integers.

Embodiments of the present invention will now be described with reference to the accompanying figures in which

FIG. 1 illustrates the process of encapsulation using an optional pretreatment step and four different encapsulation conditions;

FIG. 2 illustrates an in vitro digestion study;

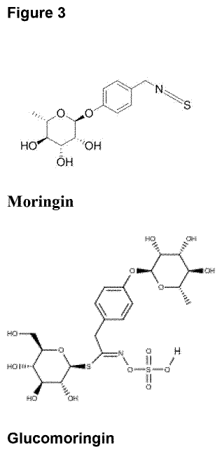

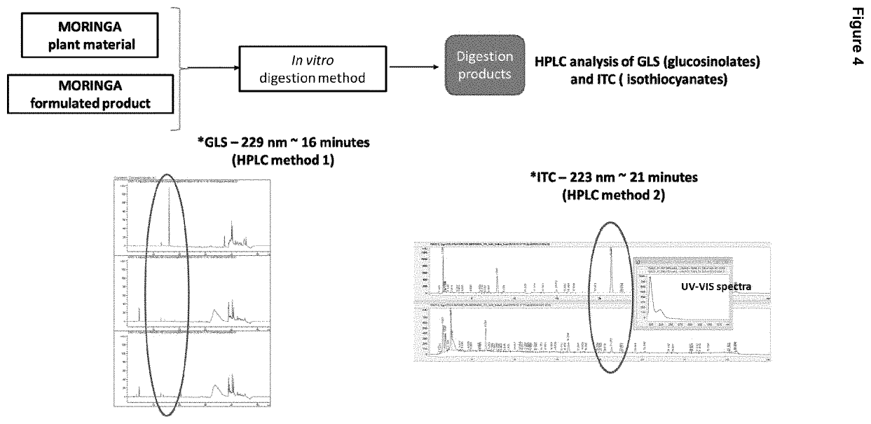

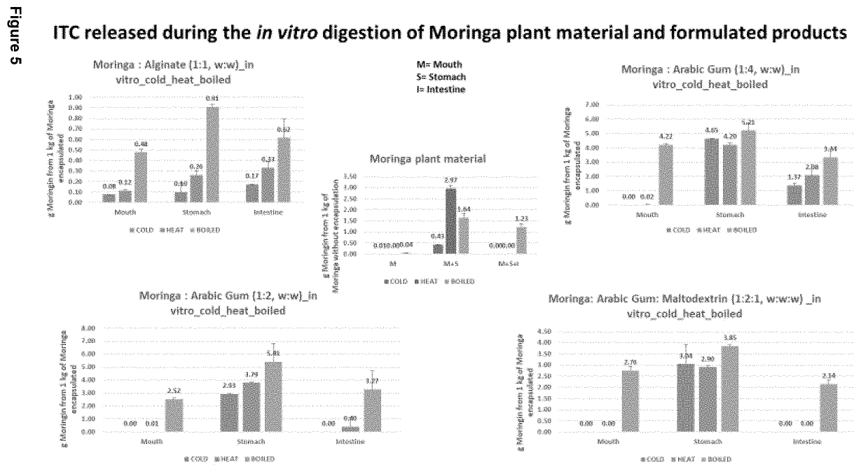

FIG. 3 illustrates glucomoringin and glocosoonjnain and conversion to isothiocyanate which provides a pungent taste to the Moringa, wherein Glucomoringin is specific for domestic Moringa and Glocosoonjnain is specific for wild type of Moringa;

FIG. 4 shows glucosinolate extraction and Isothiocyanate determination in samples using HPLC

FIG. 5 shows ITC released during the in vitro digestion of Moringa plant material and encapsulated Moringa formulated products;

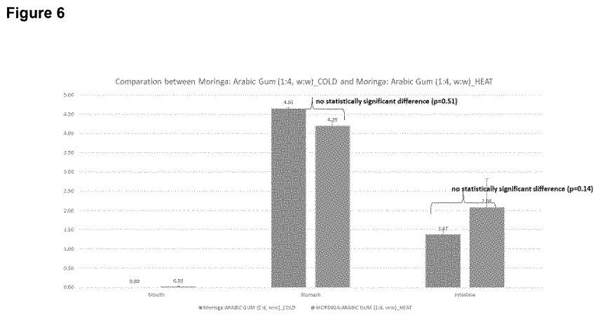

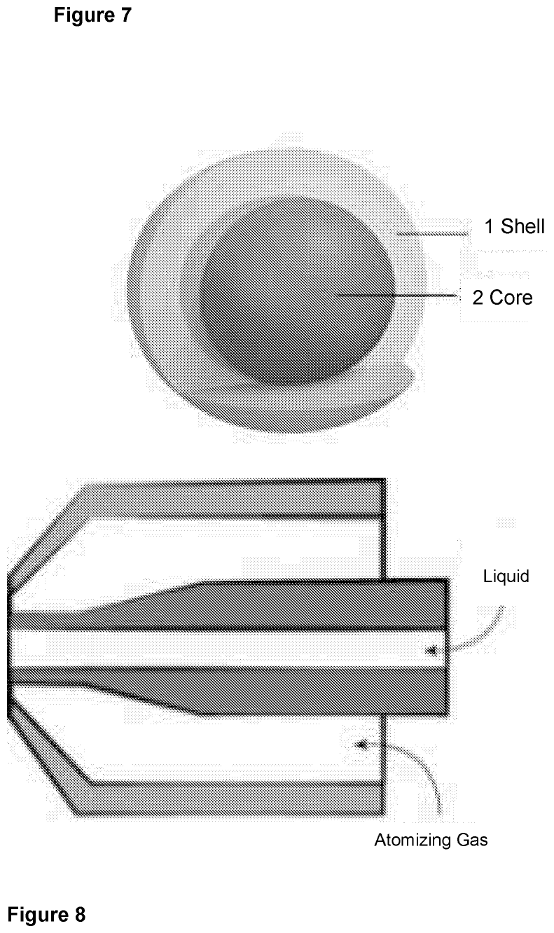

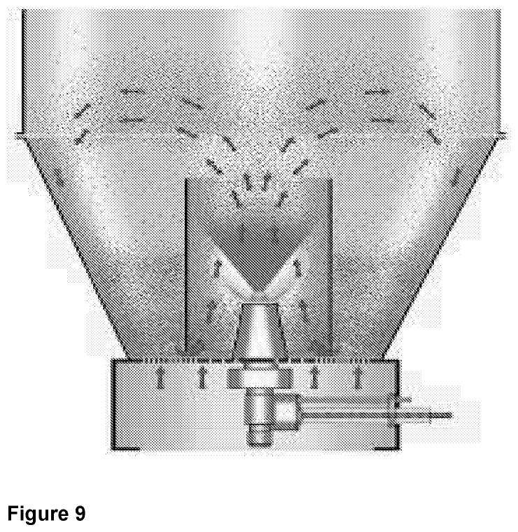

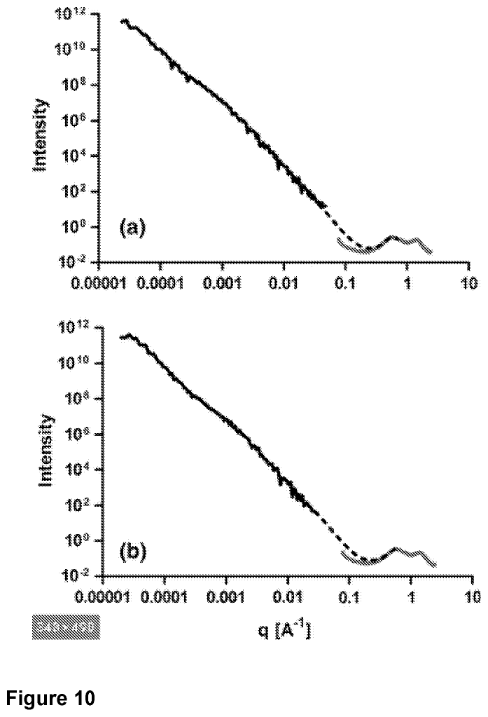

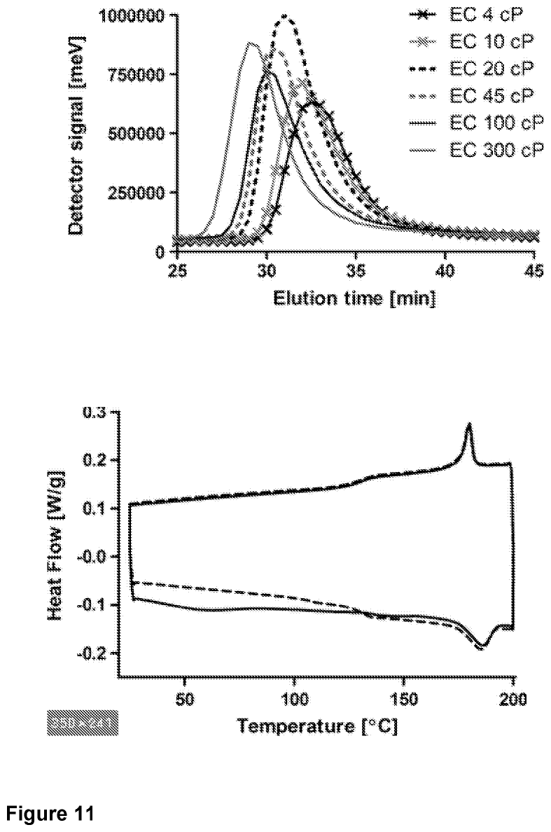

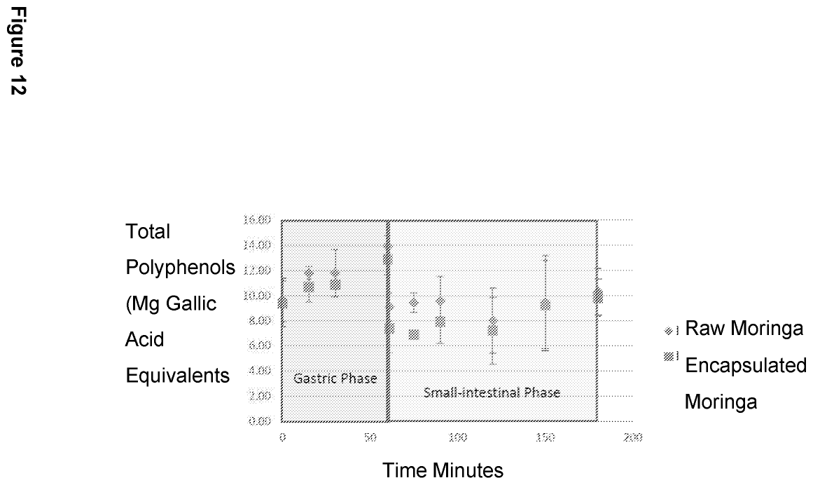

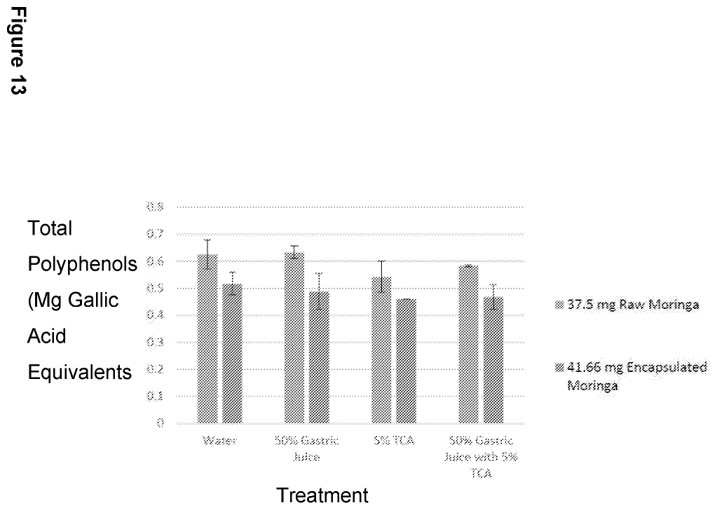

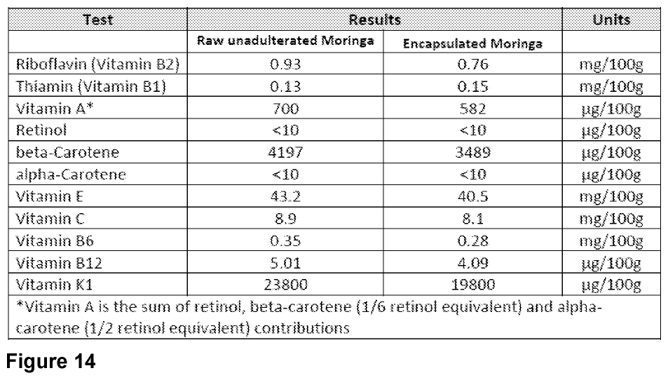

FIG. 6 shows the preferential release characteristics of a process of encapsulation using Moringa:Arabic Gum 1:4 followed by spray drying

### DETAILED DESCRIPTION

As illustrated in FIG. 1, a process to encapsulate Moringa plant material using different conditions was provided.

### Example 1

In a first process, where no heat pretreatment step is provided, (condition A), dried Moringa was milled using a ROBOQBO™ device at ambient temperature (around 20° C.). The size of the milled particle was less than 40 μm micrometers, suitably from about 2 μm to 30 μm, suitably at least 5 μm, suitably at least 10 μm, suitably at least 20 μm, suitably in a micrometer range to comply later with the encapsulation/microencapsulator nozzle diameters (i.e. 40 μm) and the milled material stored at room temperature for use as a starting material in the process of encapsulation or microencapsulation.

The Moringa was then mixed with an encapsulating agent and water, until a stable mixture/suspension was produced. A Silverson L5m-a emulsifier was used to produce the mixture.

In an example, the Moringa was mixed with sodium alginate at a ratio of 1:1 w:w until a homogenised mixture was provided. Sodium alginate is a gum, extracted from the cell walls of brown algae. It is therefore suitable for vegetarians and vegans.

Encapsulation of the Moringa with the aliginate mixture was then provided using a Biotech Encapsulator using Calcium Chloride (6%) as hardening bath. A substantially spherical product (bead) was produced and this product was left for 30 minutes in the hardening. The bead product formed was in a size range between 0.15 mm to 2.0 mm, depending of the nozzle sizes used: 0.08, 0.12, 0.15, 0.20, 0.30, 0.45, 0.75 and 1.0 mm of around. The parameters used to produce the spherical beads using Biotech encapsulator were: vibration frequency 1000 Hz and air flow rate 700 ml/h.

### Example 2

In a second process, where no heat pretreatment step is provided, (condition A), dried and milled Moringa as prepared in Example 1 was used as a starting material.

The Moringa was then mixed with an encapsulating agent and water, until a stable mixture/suspension was produced using the Silverson L5m-a emulsifier. In an example, the Moringa was mixed with Arabic Gum at a ratio of 1:4 w:w until a homogenised mixture was provided. A Silverson L5m-a emulsifier was used to produce the mixture.

The mixture was then spray dried using a Buchi Mini Spray Dryier B-290 to form a dry powder using conditions: Temperature 160° C., Pump 20%, Aspiration 100%, Flow 40-50%.

The dry powder was fine and of a lighter colour than the initial plant material to be encapsulated, with a sample size distribution between 2 and maximum 10 μm.

### Example 3

In a third process, where no heat pretreatment step is provided, (condition A), dried and milled Moringa prepared as set out in Example 1 was used as a starting material.

The Moringa was then mixed with an encapsulating agent and water, until a stable mixture/suspension was produced using the Silverson L5m-a emulsifier. In an example, the Moringa was mixed with Arabic Gum at a ratio of 1:2 w:w until a homogenised mixture was provided. A Silverson L5m-a emulsifier was used to produce the mixture.

The mixture was then spray dried using a Buchi Mini Spray Dryier B-290 to form a dry powder using conditions: Temperature 160° C., Pump 20%, Aspiration 100%, Flow 40-50%.

The dry powder was fine and of a lighter colour than initial plant material to be encapsulated.

### Example 4

In a fourth process, where no heat pretreatment step is provided, (condition A), dried and milled Moringa as discussed in Example 1 was used as a starting material. The Moringa was then mixed with an encapsulating agent and water, until a stable mixture/suspension was produced using the Silverson L5m-a emulsifier. In an example, the Moringa was mixed with Arabic Gum and Maltodextrin at a ratio of 1:2:1 w:w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%. The dry powder was fine and uniform powder, with a lighter colour than the initial initial plant material for encapsulation.

### Example 5

In a fifth process, a heat pretreatment step is provided, (condition B), where the dried and milled Moringa is subjected to heat of about 60° C. for 16 hours.

The Moringa was then mixed with an encapsulating agent and water, until a stable mixture/suspension was produced using the Silverson L5m-a emulsifier In an example of the process with a heat treatment, the Moringa was mixed with alginate as set out in Example 1 at a ratio of 1:1 w:w until a homogenised mixture was provided.

The Moringa and aliginate mixture was then provided in a Calcium Chloride (6%) hardening bath to form a substantially spherical product (bead or beads) as previously described in Example 1.

### Example 6

In a sixth process, a heat pretreatment step is provided, (condition B), where the dried and milled Moringa was subjected to heat of about 60° C. for 16 hours.

The Moringa was then mixed with an encapsulating agent and water, until a stable mixture/suspension was produced using the Silverson L5m-a emulsifier

In an example, the Moringa was mixed with Arabic Gum at a ratio of 1:4 w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%.

The dry powder was fine and of a lighter colour than initial plant material to be encapsulated, with a sample size distribution between 2 and maximum 10 μm. [

### Example 7

In a seventh process, a heat pretreatment step is provided, (condition B), where the dried and milled Moringa, was subjected to heat of about 60° C. for 16 hours. The Moringa was then mixed with an encapsulating agent and water, until a stable mixture/suspension was produced using the Silverson L5m-a emulsifier. In an example, the Moringa was mixed with Arabic Gum at a ratio of 1:2 w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%. The dry powder was fine and homogeneous powder, with a lighter colour than the initial initial plant material for encapsulation.

### 25 Example 8

In an eighth process, a heat pretreatment step is provided, (condition B), where the dried and milled Moringa, was subjected to heat of about 60° C. for 16 hours.

The Moringa was then mixed with an encapsulating agent and water, until a stable mixture/suspension was produced using the Silverson L5m-a emulsifier. In an example, the Moringa was mixed with Arabic Gum and Maltodextrin at a ratio of 1:2:1 w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%. The dry powder was fine and homogeneous powder, with a lighter colour than the initial initial plant material for encapsulation.

### Example 9

In a ninth process, a heat pretreatment step is provided, (condition C), where the dried and milled Moringa was subjected to heat treatment by mixing it with water at 100° C. and boiling it for three minutes in a water bath at 100° C.

The heat treated Moringa was then mixed with an encapsulating agent, until a stable mixture/suspension was produced using the Silverson L5m-a emulsifier. In an example of the process with a heat treatment, the Moringa was mixed with alginate at a ratio of 1:1 w:w until a homogenised mixture was provided.

The Moringa and aliginate mixture was then provided in a Calcium Chloride (6%) hardening bath to form substantially spherical product (beads) as discussed in Example 1.

### Example 10

In a tenth process, a heat pretreatment step is provided, (condition C), where the dried and milled Moringa was subjected to heat treatment by mixing it first with water at 100° C. and boiling it for three minutes in a water bath at 100° C.

The heat treated Moringa was then mixed with an encapsulating agent, until a stable mixture/suspension was produced using the Silverson L5m-a emulsifier. In an example, the Moringa was mixed with Arabic Gum at a ratio of 1:4 w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%. The dry powder was fine and homogeneous powder, with a lighter colour than the initial initial plant material for encapsulation.

### Example 11

In an eleventh process, a heat pretreatment step is provided, (condition C), where the dried and milled Moringa was subjected to heat treatment by mixing it first with water at 100° C. and boiling it for three minutes in a water bath at 100° C.

The heat treated Moringa was then mixed with an encapsulating agent, until a stable mixture/suspension was produced using the Silverson L5m-a emulsifier. In an example, the Moringa was mixed with Arabic Gum at a ratio of 1:2 w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%. The dry powder was fine and homogeneous powder, with a lighter colour than the initial initial plant material for encapsulation.

### Example 12

In a twelfth process, a heat pretreatment step is provided, (condition C), where the dried and milled Moringa was subjected to heat treatment by mixing it first with water at 100° C. and boiling it for three minutes in a water bath at 100° C. The heat treated Moringa was then mixed with an encapsulating agent, until a stable mixture/suspension was produced using the Silverson L5m-a emulsifier.

In an example, the Moringa was mixed with Arabic Gum and Maltodextrin at a ratio of 1:2:1 w:w:w until a homogenised mixture was provided.

The mixture was then spray dried using a Buchi Mini Spray Dryier B-290 to form a dry powder using conditions: Temperature 160° C., Pump 20%, Aspiration 100%, Flow 40-50%.

The dry powder was fine and homogeneous powder, with a lighter colour than the initial initial plant material for encapsulation.

### Example 13

Encapsulated plant material, for example Moringa (powder or beads) as discussed herein, encapsulated or microencapsulated as set out herein, for example as provided by any one of Examples 1 to 12 can be added to other food products and formulations and ingredients as required.

As would be appreciated in the art, the encapsulated or microencapsulated plant material, for example Moringa may be provided with flavouring, colourings or other food additives and nutrients.

Encapsulated or microencapsulated plant material, for example Moringa may also be added to other food formulation to increase the level of macronutrients, micronutrients and phytochemicals provided by the food compositions or formulations. Suitably, a food composition or formulation may be a wet or dry food formulation, for example, but not limited to, a bakery product, beverage, dairy, processed food, ready meal etc.

### Example 14

As illustrated in FIG. 2, encapsulated Moringa and non-encapsulated Moringa was tested using an in vitro digestion model to determine when the components of Moringa considered to be responsible for Moringa's pungent taste were released.

Three different in vitro conditions were used to simulate the mouth, stomach and intestine conditions of a subject that would eat the Moringa.

The mouth was simulated by providing the Moringa or encapsulated Moringa with a simulated salivary fluid (SSF) at a ratio of 1:10, w:v. The conditions used to simulate the mouth conditions were
2 minutes with SSF at 37° C., shaking in a water bath
centrifuge 4000 RPM/5 min

Samples were provided in triplicate.

Following the in vitro digestion, the digestion products were subjected to HPLC analysis.

As illustrated in FIG. 5, encapsulated Moringa shows a different release profile to Moringa when provided without encapsulation.

Without wishing to be bound by theory, it is considered heat treatment affects the microencapsulation efficiency (see boiled) and causes the encapsulation which protects the molecules, to enable release of the bioactives in the plant material later on in the GI tract.

This can be particularly advantageous as it can maximise release of the bioactive of the plant material, for example maximising moringin release. As plant bioactives, for example moringin have proven beneficial biological activity, encapsulation or microencapsulation as discussed herein can maximise the functional effect provided by the same amount of bioactive provided by a plant.

As illustrated in FIG. 6, Moringa encapsulated with Arabic Gum 1:4 after both heat and non-heat pretreatment showed similar release of ITC in the stomach and intenstine.

Although the invention has been particularly shown and described with reference to particular examples, it will be understood by those skilled in the art that various changes in the form and details may be made therein without departing from the scope of the present invention. In particular it suitably provides a method to enhance nutrient and bioactive availability and enhance the release profile of bioactives in bioactive rich plant formulation. Additionally, the method can be used to increase the palatability of bioactives.

1.-34. (canceled)

35. A process to encapsulate or microencapsulate plant material the process comprising the steps of:
mixing milled plant material with an encapsulation agent selected from:
a) a plant material: encapsulation agent at a ratio of 1:1 w/w to encapsulate the plant material; or
b) a plant material: coating for microencapsulation at a ratio of 1:2 to 1:4 w/w;
to provide a homogenized mixture wherein the plant material is encapsulated by the encapsulating agent or coated with the microencapsulation agent, and optionally further processing the homogenized mixture.

36. The process as claimed in claim 35 wherein an encapsulation agent is selected from: at least one of alginate, carrageen, cellulose sulphate, chitosan, gelatine and pectin, as well as waxes.

37. The process as claimed in claim 35 wherein the coating for microencapsulation is selected from at least one of: water soluble: polysaccharides (cellulosic and non cellulosic) and non polysaccharides, Guar gum, Carrageenan, Sodium alginate, Chitosan, Gum Arabic, Karaya gum, methylcellulose, sodium carboxylmethyl cellulose, hydroxypropyl methylcellose, Gelatin, polyvinyl alcohol, polyethylene glycol. Another category are non-water soluble coating materials:
organic soluble polymers, latex, dispersions, pseudo latex, and non-polymers.

38. A process as claimed in claim 35 to microencapsulate Moringa plant material the process comprising the steps of:
mixing milled Moringa plant material with an encapsulation agent or coating for microencapsulation selected from:
a) Alginate—at a Moringa: Alginate ratio of 1:1 w/w;
b) Arabic gum—at a Moringa: Arabic gum ratio of 1:2 to 1:4 w/w; or
c) Arabic gum maltodextrin—at a Moringa:Arabic gum:maltodextrin ratio of 1:2:1
to provide a homogenized mixture of the Moringa and the encapsulating agent or coating for microencapsulation.

39. The process of claim 35 wherein the homogenized mixture is further processed
a) wherein the homogenized mixture is a plant material: encapsulation mixture, the mixture is provided in a CaCl₂ (6%) hardening bath;
b) wherein when a plant material: coating for microencapsulation is provided the homogenized mixture is spray dried to provide a powder.

40. The process of claim 35 wherein the homogenized mixture is further processed
a) wherein the homogenized mixture is a Moringa: Alginate mixture, the mixture is provided in a CaCl₂) (6%) hardening bath;
b) wherein when a Moringa: arabic gum mixture, or a Moringa: arabic gum: Maltodextrin mixture is provided the homogenized mixture is spray dried to provide a powder.

41. The process of claim 35 wherein the Moringa used in the process is whole plant

42. The process of claim 35 wherein the process includes a pre-treatment step, after milling and before mixing.

43. The process of claim 42 wherein the pre-treatment step comprises heating of the milled material to 60° C. for 16 hours in an oven.

44. The process of claim 43 wherein the pre-treatment step comprises providing hot water to the milled material, optionally Moringa, for 3 minutes in a water bath at 100° C.

45. A nutritional composition comprising encapsulated plant material or microencapsulated plant material as provided by the process of:
mixing milled plant material with an encapsulation agent selected from:
c) a plant material: encapsulation agent at a ratio of 1:1 w/w to encapsulate the plant material; or
d) a plant material: coating for microencapsulation at a ratio of 1:2 to 1:4 w/w;
to provide a homogenized mixture wherein the plant material is encapsulated by the encapsulating agent or coated with the microencapsulation agent, and optionally further processing the homogenized mixture.

46. A nutritional composition comprising encapsulated Moringa or microencapsulated Moringa as provided by the process of:
mixing milled plant material with an encapsulation agent selected from:
e) a plant material: encapsulation agent at a ratio of 1:1 w/w to encapsulate the plant material; or
f) a plant material: coating for microencapsulation at a ratio of 1:2 to 1:4 w/w;
to provide a homogenized mixture wherein the plant material is encapsulated by the encapsulating agent or coated with the microencapsulation agent, and optionally further processing the homogenized mixture.
